(19) United States
(12) Patent Application Publication
(10) Pub. No.: US 2002/0169565 A1
(43) Pub. Date: Nov. 14, 2002

(54) System and method for data deposition and annotation

(76) Inventors: John D. Westbrook, Highland Park, NJ (US); Helen M. Berman, Lawrenceville, NJ (US)

Correspondence Address: Mathews, Collins, Shepherd & McKay, P.A., Suite 306, 100 Thanet Circle, Princeton, NJ, 08540, US

(21) Appl. No.: 10/132,627

(22) Filed: Apr. 24, 2002

Related U.S. Application Data
(60) Provisional application No. 60/286,255, filed on Apr. 25, 2001.

(51) Int. Cl. G01N 33/50; G06F 19/00

(52) U.S. Cl. 702/20; 702/27; 707/104.1

(57) ABSTRACT

The present invention provides an integrated data system for processing deposition and annotation of data such as three dimensional macromolecular structure data. The system is based on a new community data standard referred to as meta data. The meta data structure provides data or information about other data. Unlike the previous PDB data format, in the present invention PDB both the syntax and semantics of the PDB data standard are rigorously defined and encoded in meta data dictionaries which are fully software accessible. The data processing system of the present invention uses meta data at every functional step beginning with data collection.

### BACKGROUND OF THE INVENTION

1. Field of the Invention

The present invention relates to a system and method for data deposition, data processing and annotation which can be used with three dimensional macromolecular structure data.

2. Description of Related Art

For the past 25 years the Protein Data Bank (PDB) has served as the single central repository for macromolecular structure data. During the first two decades of operation, the PDB was managed by Brookhaven National Laboratories (BNL). In the early history of the PDB structure data was deposited in a variety of media: paper hardcopy, magnetic tape, and diskette. In the latter years of BNL operation data was also collected through a web-based interface. This deposition interface was supported by a collection of Perl scripts individually tailored to provide data input forms corresponding to the PDB data file format.

The PDB data format is a column-oriented data format resembling the typical many data formats developed to accommodate the limitations of paper punch card technology. An example of the data format is shown in FIG. 1. Many of the data records in the format shown in FIG. 1 are prefixed with a record tag (e.g. CRYST1, ATOM) followed by individual items or data. The specifications for this data format are described informally in the PDB Content Guide: Atomic Coordinate Entry Format Description as described in http://www.rcsb.org/pdb/docs/format/pdbguide2.2/guide2.2 frame.html. In addition to the labeled records like those in FIG. 1, many data records in the PDB format are presented as unstructured or only semi-structured remark records.

It is desirable to provide an improved system and method for deposition and annotation of macromolecular structure data which system can also be used for deposition and annotation of any content domain.

### SUMMARY OF THE INVENTION

The present invention provides an integrated data system for processing deposition and annotation of data such as three dimensional macromolecular structure data. The system is based on a new community data standard referred to as meta data. The meta data structure provides data or information about other data. Unlike the previous PDB data format, in the present invention PDB both the syntax and semantics of the PDB data standard are rigorously defined and encoded in meta data dictionaries which are fully software accessible.

An important end result of the data processing of PDB data in the present invention is the production of uniform archival data files and a database resource that is broadly useful to researchers in structural biology. The database resource is sufficiently well described that it can be easily integrated with other chemical and biological databases. Meta data dictionaries are used in the present invention as key components for developing an infrastructure to support systematic analyses of diverse data resources. Any dictionary which complies with the dictionary description language, such as DDL2, can be loaded and used by the system of the present invention. The metadata description provides precise definitions and detailed attributes for each item of data which description allows the data to be reliably queried and compared within and across databases.

The data processing system of the present invention uses metadata at every functional step beginning with data collection. Applying the content of the data dictionary in a consistent manner at each stage of data processing and annotation helps to achieve uniformity and reliability useful in the database end product. All of the software components gain their knowledge of the input data from the data dictionary and any associated data views of the present invention. Accordingly, the system of the present invention can be used for virtually any data input and data processing application. The present invention provides flexible and extensible data processing features by exploiting the features of this general metadata framework. The invention will be more fully described by reference to the following drawings.

### BRIEF DESCRIPTION OF THE DRAWINGS

FIG. 1 is a schematic diagram of a record from a prior art protein data bank data file.

FIG. 2 is a schematic diagram of a system for data deposition, data processing and annotation in accordance with the teachings of the present invention.

FIG. 3 is a schematic diagram of an implementation of an auto deposition input tool used in the system of the present invention in accordance with the teachings of the present invention.

FIG. 4 is a schematic diagram of an excerpt of a macromolecular crystallographic information file (mmCIF) of an individual file.

FIG. 4B is a schematic diagram of an excerpt of a macromolecular crystallographic information file (mmCIF) describing the individual data files in a data dictionary definition.

FIG. 4C is a schematic diagram of an excerpt of a macromolecular crystallographic information file (mmCIF) described in a data description language.

FIG. 5 is a schematic diagram of an example data input screen in accordance with the teachings of the present invention.

FIG. 6 is a schematic diagram of the system of the present invention including a database loader.

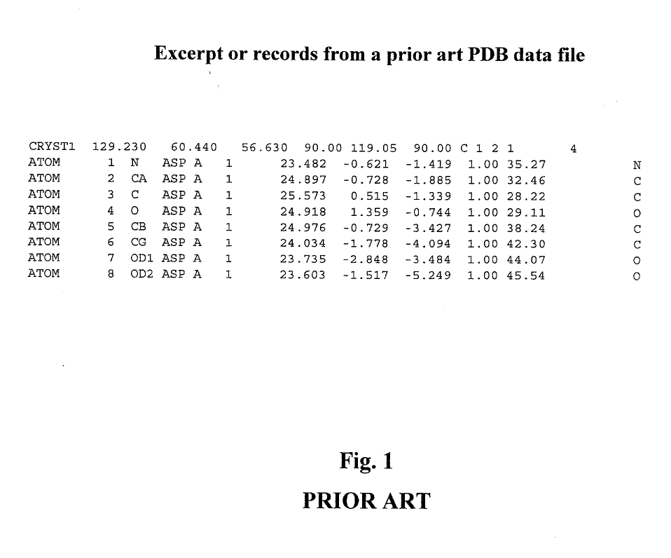

### DETAILED DESCRIPTION

Reference will now be made in greater detail to a preferred embodiment of the invention, an example of which is illustrated in the accompanying drawings. Wherever possible, the same reference numerals will be used throughout the drawings and the description to refer to the same or like parts.

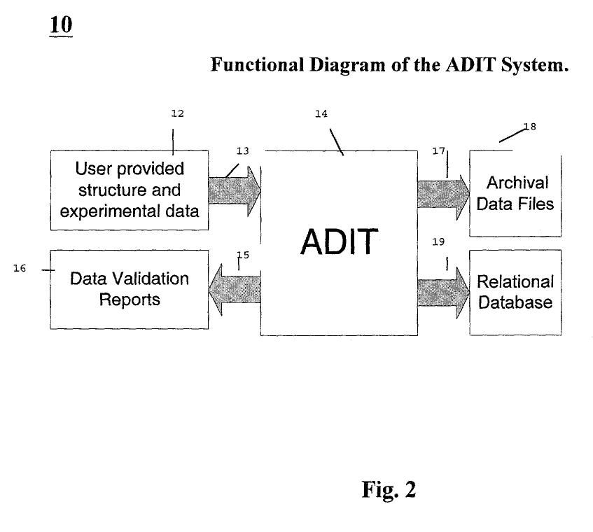

FIG. 2 illustrates a schematic diagram of a system for data deposition, data processing and annotation 10 in accordance with the teachings of the present invention. In block 12, experimental and structural data are input from a depositing user as input data 13. Input data 13 is input either from in the form of data files or through a web-based form interface.

Input data 13 is received at auto deposition input tool (ADIT). For example, input data 13 can relate to macromolecular structure data including atomic coordinate data, genome information for the deposited structures and information specific to the method of structure determination as deposited in the PDB. Alternatively, input data 13 can be data in any content domain. Input data 13 can be validated by ADIT 14 in a very basic sense for syntax compliance and internal consistency. Other computational validation can also be applied: such as for example checking the input structure data against a variety of community standard geometrical criteria and comparing the input experimental data with the derived structure model. Validation information 15 created by ADIT 14 is returned to the user in block 16 as a collection of data validation reports. For example data validation reports can be HTML reports.

Other outputs of ADIT 14 include data encoded in archival data files 17 which can be archived in block 18. Outputs of ADIT 14 can be annotated to form annotated output 19 and loaded into a relational database in block 20. Annotated output 19 can be determined with an expert annotator. ADIT 14 adapts to the requirements of its user and customizes its behavior according to the users requirements. For example a depositing user and an expert annotator user can provide different data input. In general a depositing user is focused only on data collection and provides the simplest possible presentation of the information to be input. The expert user sees a detail of all possible data input as well as the full functionality of the supporting data processing and database system.

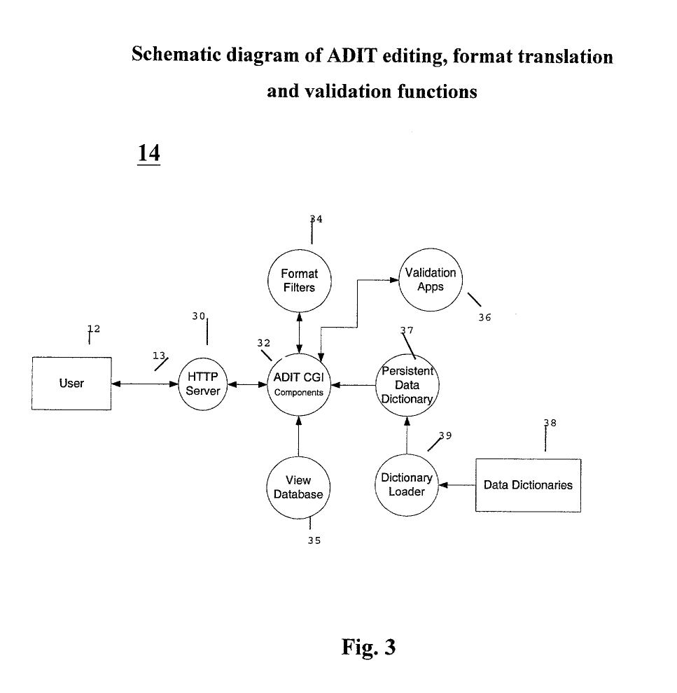

FIG. 3 illustrates an implementation of ADIT 14. Users in block 12 interact with ADIT 14 through web server 30. User of block 12 interfaces with Interface 32. Interface 32 can be a common gateway interface (CGI). CGI components can dynamically build HTML to provide a system user interface which can be accessed through web server 30. The CGI components can be implemented for example as compiled binaries from C++ source code. Alternatively, interface 32 can be a server oriented architecture implemented using servlets instead of CGI components.

Input data 13 can be provided in the form of data files or as keyboard input by a user in block 12. Files can be accepted in a variety of formats. Format filters 34 convert input data 13 to the data specification defined in a persistent data dictionary 37. Input data 13 in the form of data files is typically loaded first. Any input data 13 that is not included in uploaded files can be keyed in by the user. For example, format filters 34 can build a set of HTML forms for each category of data to be input. At any point a user any choose to view or deposit contents of input data 13 through interface 32. Users in block 12 can also execute data validation applications services 36.

Data dictionaries 38 provide a description of any type of data. Data dictionaries can preferably be developed as meta data. Meta data can be defined as data or information about other data. For example, data dictionaries 38 can provide a comprehensive ontology of experimental crystallography and macromolecular structure, as described in detail below.

View database 35 is used for selecting only the relevant set of input data items from a data dictionary 38. A data view is used to define the scope of the data items to be edited by the ADIT, and to store presentation details that are used in building the HTML input forms. The data view provides a simple and intuitive presentation of information for novice users. This is often useful in order to disguise the complex details of a data dictionary.

Dictionary loader 39 provides efficient access to attributes from data dictionaries 38. For example, dictionary loader 39 can provide tabular text structure to an object representation. The class supporting the object representation provides efficient access functions to all of the data dictionary attributes. Dictionary loader 39 can be used to check the consistency of data dictionary 38 and load the object representation from the text form of data dictionary 38 for determining information of attributes from data dictionaries 38. Persistent data dictionary 37 provides loading of the object of dictionary loader 39 from a storage medium.

In a preferred embodiment data dictionaries 38 are generated in a meta data architecture to define crystallography and macromolecular structure. For macromolecular applications an ontology has been represented in a conventional Macromolecular Crystallographic Information File (mmCIF) data dictionary using a self-defining text archival and retrieval syntax (STAR). The mmCIF data dictionary, was developed within the crystallographic community under the auspices of the International Union of Crystallography (IUCr) as described in Bourne et al., Methods Enzymol., 277,571-590 (1997). MmCIF is used as the standard data representation for experimentally determined 3D macromolecular structures.

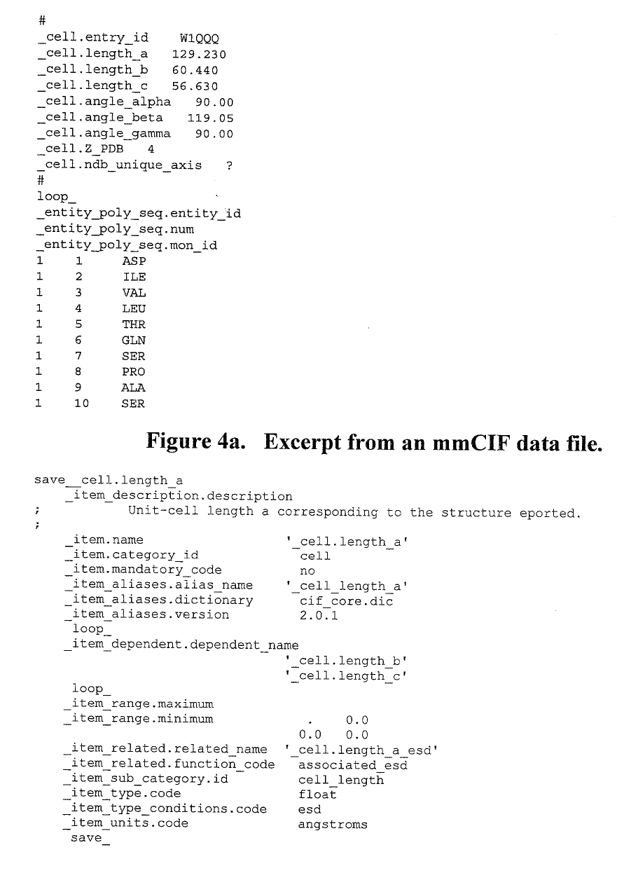

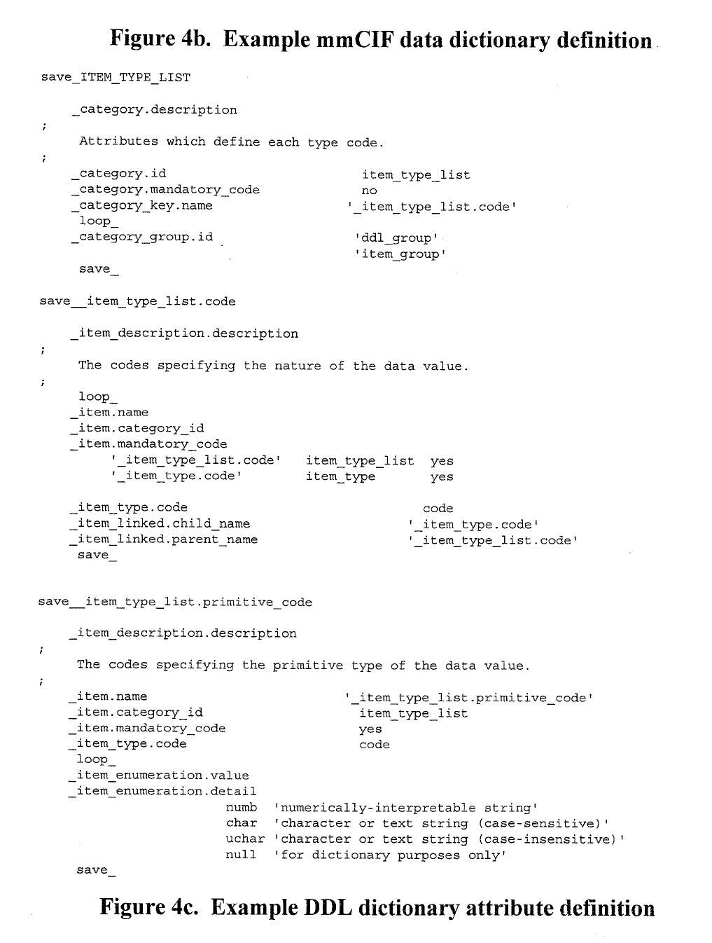

In this embodiment the mmCIF metadata architecture is built from three levels as shown in FIGS. 4a-c. Individual data files are described at the top level, shown in FIG. 4a. The contents of these data files are defined by the data dictionary in the next lower level, shown in FIG. 4b. The attributes used in this data dictionary to build data definitions are in turn defined in the dictionary description language (DDL) in the lowest level, shown in FIG. 4c.

The major syntactical constructs used by mmCIF are illustrated by the data file example in FIG. 4a. Each data item or group of data items is preceded by an identifying keyword. Groups of related data items are organized in data categories. Two categories, CELL and ENTITY_POLY are shown in the example. The former contains an individual instance describing a single set of crystallographic cell constants. The latter contains a loop_ (i.e. table) of instances describing a polymer residue sequence. Essentially all mmCIF data is described in tabular data structures, or as the special case of a table with unit cardinality.

Each mmCIF data item is defined in a data dictionary 28 using meta data. Data definitions are encapsulated between save frame delimiters (i.e. save_); otherwise, the data definitions share the same simple syntax as used in data files. An example definition for a crystallographic cell constant is show in FIG. 4b. Many features of the cell constant are described in this definition, including: data type, range restrictions, units of expression, dependent quantities, related definitions, necessity, and related precision estimate. Although not shown in this example, dictionary definitions can also include parent-child relationships which have important consequences in maintaining data consistency.

The attributes of each data definition are defined in a dictionary description language (DDL). FIG. 4c shows example DDL definitions describing data types using meta data. DDL definitions have the same syntax as definitions used in the data dictionary. Because the attributes of the DDL are also used in DDL definitions this meta data architecture is described as self-defining.

Comprehensive data dictionaries like mmCIF contain vast numbers of data definitions. A data input application may only need to access a small fraction of these definitions at any point. View database 35 can be used for selecting relevant items of the mmCIF dictionary defined as meta data.

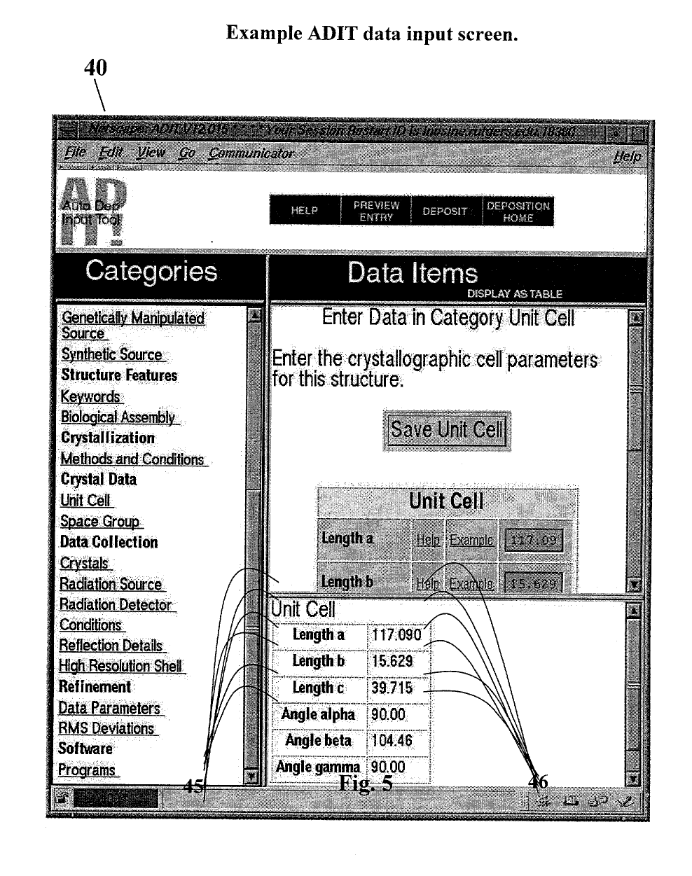

FIG. 5 shows an example data input screen 40 generated by data dictionary interface 32 for a crystallographic unit cell. Data input screen 40 includes categories 41. In this example, the data dictionary category containing this information is named, cell, and the length of the first cell axis is defined in the dictionary as_cell, length_a as defined in FIG. 3b. In this case the data view has substituted, Unit Cell 41, Length a 42 and Length b 43 for the more cryptic data names defined in data dictionary 38. Although this example is quite simple some dictionary data names are as long as 75 characters, and in these instances the ability to display a simpler name is essential.

Precise dictionary definitions and examples are accessible on data input screen 40 from buttons 45 displayed adjacent to each data item. Displayed data 46 is obtained from data dictionary 38. Accordingly, the system of the present invention makes full use of the dictionary specification in data input operations. Preferably data items which are defined to assume only specific values are presented as pull down menus or selection boxes in data input screen 40. Data type and range restrictions are checked when data are input and diagnostics are displayed to the user if errors are detected.

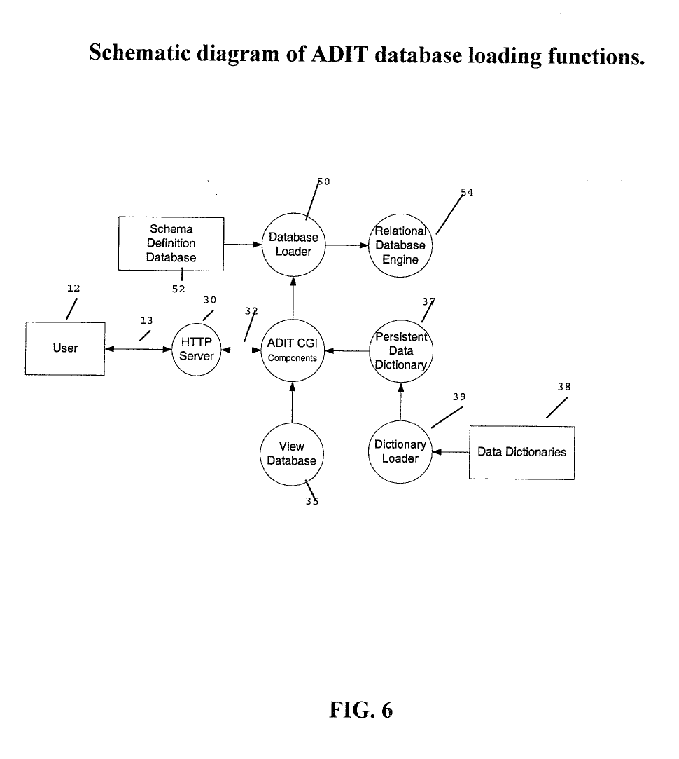

FIG. 6 illustrates an embodiment of system 10 including database loader 50. Database loader 50 can be used to build database schemas, and extract processed data required to load database instances. Schemas are defined in a meta data repository in block 52 which is accessed by the database loader 50. In the simplest case, a schema can be constructed which is modeled directly from data dictionary 38. The data model underlying the dictionary description language used to build data dictionaries 38 is essentially relational such that mapping a data dictionary specification to a relational schema can be straightforwardly performed in relational database engineering with relational database engine 54.

In other cases, a mapping is required between the target schema and the data dictionary specification of block 52. This mapping is encoded in the schema metadata repository. Database loader 50 uses this mapping information to extract items from data files and translate this data into a form which can be loaded into the target database schema. The definition of the mapping operation can include: selection operations with equijoin constraints (e.g. the value of _entity.type where_entity.id=1), aggregation (e.g. count, sum, average, collapse (e.g. vector to string)), type conversions, and existence tests.

Schema definitions are converted by database loader 50 into structural query language (SQL) instructions which create the defined tables and indices. Loadable data is produced either as XML, SQL insert/update instructions or in the table copy formats used by database engines such as Sybase, Oracle or MySQL.

It is to be understood that the above-described embodiments are illustrative of only a few of the many possible specific embodiments which can represent applications of the principles of the invention. Numerous and varied other arrangements can be readily devised in accordance with these principles by those skilled in the art without departing from the spirit and scope of the invention.

What is claimed is:

1. A method for processing data comprising the steps of:
receiving input data related to macromolecular structure data;
converting said received input data into a data specification of a persistent data dictionary defining crystallography and macromolecular structure using meta data;
depositing said data specification into an archival data file; and
archiving said archival data file.

2. The method of claim 1 further comprising viewing items of said data specification from said archival data file.

3. The method of claim 1 further comprising the steps of:
annotating said received input data to form annotated output data, said step of annotating said received input data being performed in parallel with said step of converting said received input data; and
storing said annotated output data.

4. The method of claim 3 wherein said annotated output data is stored in a relational database.

5. The method of claim 1 wherein said step of receiving input data comprises a user interface including one or more HTML forms for each category of said input data.

6. The method of claim 1 wherein said input data is a data file.

7. The method of claim 1 wherein said input data is selected from the group consisting of atomic coordination data, genome information and structure determination information.

8. The method of claim 1 wherein said persistent data dictionary is defined in a dictionary description language.

9. The method of claim 1 wherein said persistent data dictionary is a macromolecular crystallographic information file (mmCIF) data dictionary represented by meta data.

10. The method of claim 1 further comprising the step of loading one or more data dictionaries into said persistent data dictionary, said one or more data dictionaries being defined in meta data.

11. The method of claim 1 wherein said data specification describes an attribute of a crystallographic cell constant.

12. The method of claim 1 wherein said persistent data dictionary is represented by a database schema including meta data corresponding to a data dictionary.

13. The method of claim 12 further comprising a mapping between said database schema and said data dictionary.

14. A system for processing data comprising the steps of:
means for receiving input data related to macromolecular structure data;
means for converting said received input data into a data specification of a persistent data dictionary defining crystallography and macromolecular structure using meta data;
means for depositing said data specification into an archival data file; and
means for archiving said archival data file.

15. The system of claim 14 further comprising:
means for viewing items of said data specification from said archival data file.

16. The system of claim 14 further comprising:
means for annotating said received input data to form annotated output data; and
means for storing said annotated output data.

17. The system of claim 16 wherein said annotated output data is stored in a relational database.

18. The system of claim 14 wherein said means for receiving input data comprises a user interface including one or more HTML forms for each category of said input data.

19. The system of claim 14 wherein said input data is a data file.

20. The system of claim 14 wherein said input data is selected from the group consisting of atomic coordination data, genome information and structure determination information.

21. The system of claim 14 wherein said persistent data dictionary is defined in a dictionary description language.

22. The system of claim 14 wherein said persistent data dictionary is a macromolecular crystallographic information file (mmCIF) data dictionary represented by meta data.

23. The system of claim 14 further comprising a dictionary loader which loads one or more data dictionaries into said persistent data dictionary, said one or more data dictionaries being defined in meta data.

24. The system of claim 14 wherein said data specification describes an attribute of a crystallographic cell constant.

25. The system of claim 14 wherein said persistent data dictionary is represented by a database schema including meta data corresponding to a data dictionary.

26. The system of claim 25 further comprising a mapping between said database schema and said data dictionary.

27. A method for processing data comprising the steps of:
receiving input data;
converting said received input data into a data specification of a persistent data dictionary using meta data;
depositing said data specification into an archival data file; and
archiving said archival data file.

28. The method of claim 27 further comprising viewing items of said data specification from said archival data file.

29. The method of claim 27 further comprising the steps of:
annotating said received input data to form annotated output data, said step of annotating said received input data being performed in parallel with said step of converting said received input data; and
storing said annotated output data.

30. The method of claim 29 wherein said annotated output data is stored in a relational database.

31. The method of claim 27 wherein said step of receiving input data comprises a user interface including one or more HTML forms for each category of said input data.

32. The method of claim 27 wherein said persistent data dictionary is defined in a dictionary description language.

33. The method of claim 27 further comprising the step of loading one or more data dictionaries into said persistent data dictionary, said one or more data dictionaries being defined in meta data.

34. The method of claim 27 wherein said persistent data dictionary is represented by a database schema including meta data corresponding to a data dictionary.

35. The method of claim 34 further comprising a mapping between said database schema and said data dictionary.

35. A system for processing data comprising the steps of:
means for receiving input data;
means for converting said received input data into a data specification of a persistent data using meta data;
means for depositing said data specification into an archival data file; and
means for archiving said archival data file.

36. The system of claim 35 further comprising means for viewing items of said data specification from said archival data file.

37. The system of claim 35 further comprising:
means for annotating said received input data to form annotated output data, said step of annotating said received input data being performed in parallel with said step of converting said received input data; and
means for storing said annotated output data.

38. The system of claim 37 wherein said annotated output data is stored in a relational database.

39. The system of claim 35 wherein said means for receiving input data comprises a user interface including one or more HTML forms for each category of said input data.

40. The system of claim 35 wherein said persistent data dictionary is defined in a dictionary description language.

41. The system of claim 38 further comprising a dictionary loader which loads one or more data dictionaries into said persistent data dictionary, said one or more data dictionaries being defined in meta data.

42. The system of claim 35 wherein said persistent data dictionary is represented by a database schema including meta data corresponding to a data dictionary.

43. The system of claim 42 further comprising a mapping between said database schema and said data dictionary.
